(19) United States
(12) Patent Application Publication
(10) Pub. No.: US 2020/0098345 A1
(43) Pub. Date: Mar. 26, 2020

(54) SOUND AMPLIFICATION STETHOSCOPES

(71) Applicant: Donald Lynn Loriaux, West Linn, OR (US)

(72) Inventor: Donald Lynn Loriaux, West Linn, OR (US)

(21) Appl. No.: 16/142,663

(22) Filed: Sep. 26, 2018

(51) Int. Cl. G10K 11/08; A61B 7/02

(52) U.S. Cl. CPC G10K 11/08; A61B 7/02

(57) ABSTRACT

A method, system, apparatus, and/or device for capturing sounds from an appendage and/or organ of a body. The method, system, apparatus, and/or device may include a bell configured to capture a low-frequency vibration from a surface of a body. The bell may include a first base having a first diameter, a first tapered portion extending upwardly from the first base and tapering inwardly, a first top portion having a second diameter and connected to a top of the first tapered portion, and a first channel extending from the first tapered portion to the hollow portion of the first tapered portion. The first base, the first tapered portion, and the first top portion may form an elongated cone shape.

### BACKGROUND

Doctors, paramedics, nurses, and other medical professionals use stethoscopes to transmit sounds from an appendage or organ of a patient to the ears of the medical professional. For example, a medical professional may use a stethoscope to listen to heart sounds of a patient via a column of air of a stethoscope that conducts sound from the appendage or organ of the patient to the medical professional's ears. Stethoscopes are used to evaluate the cardiac and respiratory systems by allowing the user to distinguish the sounds that the beating heart generates and as the sounds of gas exchange in the lungs. The quality of the sounds denotes whether an organ or appendage of the patient being examined is healthy or unhealthy.

### BRIEF DESCRIPTION OF THE DRAWINGS

The present description will be understood more fully from the detailed description given below and from the accompanying drawings of various embodiments of the present embodiment, which is not to be taken to limit the present embodiment to the specific embodiments but are for explanation and understanding.

FIG. 1 illustrates a side perspective view of a stethoscope, according to an embodiment.

FIG. 2A illustrates a side perspective view of the bell of the stethoscope in FIG. 1, according to an embodiment.

FIG. 2B illustrates a bottom perspective view of the bell, according to an embodiment.

FIG. 2C illustrates a side perspective view of the bell, according to an embodiment

FIG. 2D illustrates a cross-sectional view of the bell of the stethoscope in FIG. 1, according to an embodiment.

FIG. 3A illustrates a top view of the diaphragm in FIG. 1, according to an embodiment.

FIG. 3B illustrates a side view of the diaphragm with a disc, according to an embodiment.

FIG. 3C illustrates a side view of the diaphragm with a tapered cavity, according to an embodiment.

FIG. 3D illustrates a side exposed view of the diaphragm with a retainer ring, according to an embodiment.

FIG. 4 illustrates the junction piece connected to the tubing, the bell, and the diaphragm, according to an embodiment.

### DETAILED DESCRIPTION

The disclosed sound amplification stethoscopes will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, a variety of sound amplification stethoscopes examples are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

A conventional acoustic stethoscope generally includes a flexible rubber tube connected to earpieces at one end and a bell with a diaphragm at the other end. The conventional bell and diaphragm capture and transmit sound from a patient's organ or appendage via a column of air in the tube to the earpieces. Conventionally, the bell is configured to capture low-pitch sounds (such as a heartbeat) and the diaphragm is configured to capture high pitch sounds (such as lung sounds). To capture the sound from the patient, the bell and the diaphragm vibrate at different frequencies.

The vibrations then move up and down the column of air inside the stethoscope tube, which in turn moves air in and out of the medical professional's ear canal and allows the medical professional to hear the captured sound. However, the conventional bells and diaphragms are ineffective at capturing many lower frequency sounds and higher frequency sounds because of the sizes, shapes, or materials of the bells and diaphragms. When the conventional bells and diaphragms ineffectively or incorrectly capture the sounds, a medical professional may be unable to diagnose an ailment of a patient or may misdiagnose the ailment because the medical professional may not hear the correct sounds from the patient's appendage or organ.

Implementations of the disclosure address the above-mentioned deficiencies and other deficiencies by providing a method, system, device, and/or apparatus to capture sounds from a patient's appendage or organ. The method, system, device, or apparatus may utilize a wooden bell with a defined shape and size to capture the low-frequency sounds that are then relayed to the medical professional by an attached tube and earpieces. The method, system, device, or apparatus may utilize a diaphragm with a defined shape and size to capture the high-frequency sounds that are then relayed to the medical professional by the tube and earpieces. An advantage of the wooden bell and the diaphragm are to accurately capture low-frequency and/or high-frequency sounds from a patient so that a medical professional may accurately diagnose an ailment. Another advantage of the wooden bell and/or the diaphragm are that they may be relatively small, such that the medical professional may easily carry the bell, the diaphragm, and/or stethoscope as needed.

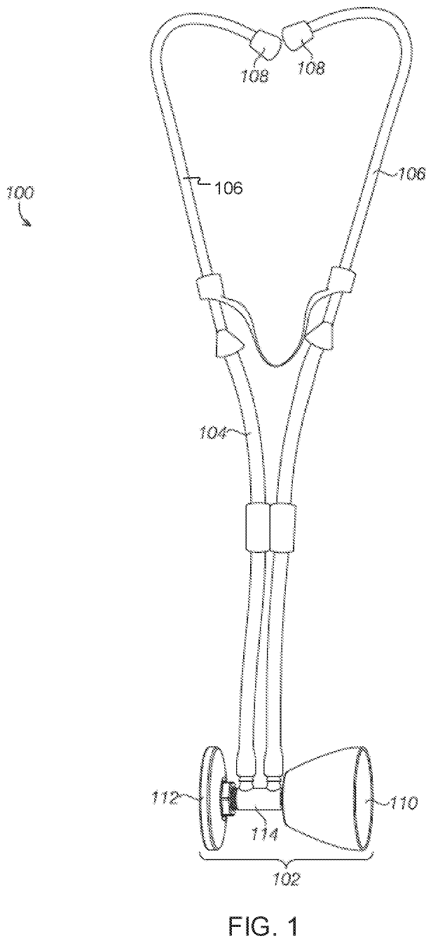

FIG. 1 illustrates a side perspective view of a stethoscope 100, according to an embodiment. The stethoscope 100 may include a listening apparatus 102, tubing 104, a binaural apparatus 106, and earpieces 108. The listening apparatus 102 may be connected to a first end of the tubing 104 and a first end of the binaural apparatus 106 may be connected to a second end of the tubing 104. In one embodiment, the tubing 104 may be a soft flexible material to allow the tubing 104 to flex. In another embodiment, the tubing 104 may be a hard rubber or plastic material that may transfer the sound vibrations from the listening apparatus 102 to the earpieces 108. A second end of the binaural apparatus 106 may be connected to the earpieces 108.

The listening apparatus 102 may be configured to be placed approximate to or against a portion of a patient's body to listen to the appendage or organ of the patient. The listening apparatus 102 may include a bell 110 and/or a diaphragm 112. In one embodiment, the bell 110 may be configured to selectively transmit low-frequency sounds. In another embodiment, the bell 110 may be configured to capture low-frequency sounds and high-frequency sounds but low-frequency sounds from the patient may mask the high-frequency sounds such that the high-frequency sounds may not be audible to the medical professional. In one embodiment, the low-frequency sounds may range from 1-200 hertz (Hz). In another embodiment, the low-frequency sounds may range from 20-650 hertz (Hz). In another embodiment, the low-frequency sounds may include a frequency range for sounds made by a heart. In another embodiment, the low-frequency sounds may include heart sounds and lung sounds for frequencies lower than 1000 cycles per second.

In another embodiment, the high-frequency sounds may range from 200-1500 Hz. In another embodiment, the high-frequency sounds may range from 20-2000 Hz. For example, the diaphragm 112 may pick-up the low-frequency sounds of 20-200 Hz and the high-frequency sounds of 200-1500 Hz, but the diaphragm 112 may drop barely audible or inaudible low-frequency sounds below 200 Hz. In another embodiment, the high-frequency sounds may include a range for sounds made by a lung or lungs.

In another embodiment, the diaphragm 112 may selectively filter out low-frequency sounds such that high-frequency sounds may be transmitted via the tubing 104 and the binaural apparatus 106 to the earpieces 108. In another embodiment, the diaphragm 112 may not filter out low-frequency sounds and may attenuate all frequencies substantially the same so as to drop barely audible or inaudible low-frequency sounds below the threshold of human hearing.

In one embodiment, a frequency range of the bell 110 may be modified or adjusted based on an amount of pressure used by a medical professional when pressing the bell 110 against the body or skin of a patient. For example, when the medical professional applies relatively no pressure to the bell 110 when the bell 110 contacts the skin of a patient, the bell 110 may pick up a first low-frequency range of 20-650 Hz. In another example, when the medical professional applies a relatively high amount of pressure to the bell 110 when the bell 110 contacts the skin of a patient, the bell 110 may pick up the a second frequency range of 1-200 Hz. In another example, the medical professional may apply relatively no pressure to the bell 110 when examining the lungs of a patient such that the bell 110 may capture sounds from the lung with a frequency of 20-650 Hz. In another example, the medical professional may apply the relatively high amount pressure to the bell 110 when examining the heart of a patient such that the bell 110 may capture sounds from the lung with a frequency of 1-200 Hz and filter out sounds from the heart of the patient.

In one embodiment, a frequency range of the diaphragm 112 may be modified or adjusted based on an amount of pressure used by a medical professional when pressing the diaphragm 112 against the body or skin of a patient. For example, when the medical professional applies relatively no pressure to the diaphragm 112 when the diaphragm 112 contacts the skin of a patient, the diaphragm 112 may pick up a first high-frequency range of 20-2000 Hz. In another example, when the medical professional applies a relatively large amount of pressure to the diaphragm 112 when the diaphragm 112 contacts the skin of a patient, the diaphragm 112 may pick up a second frequency range of 650-2000 Hz. In another example, the medical professional may apply relatively no pressure to the diaphragm 112 when examining the lungs of a patient such that the diaphragm 112 may hear sounds from the heart with a frequency of 20-2000 Hz. In another example, the medical professional may apply the relatively large amount pressure to the diaphragm 112 when examining the heart of a patient such that the diaphragm 112 may hear sounds from the lung with a frequency of 650-2000 Hz in order to filter out sounds from the heart of the patient.

In one embodiment, the low amount of pressure may be less than 0.4 Newtons of force and the high amount of pressure may be equal to or greater than 0.4 Newtons of force. In another embodiment, a medical professional may vary the amount of pressure or force until the medical professional may hear a desired sound from the organ or appendage with the highest amount of sound clarity relative to the sound clarity for other amounts of pressure or force.

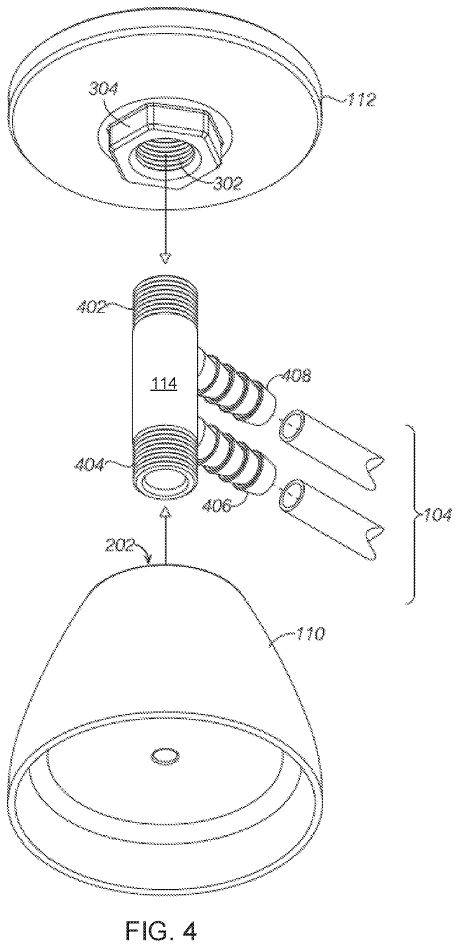

As discussed below for FIG. 4, the junction piece 114 may connect the bell 110 and/or the diaphragm 112 to the tubing 104 such that the bell 110 and/or the diaphragm 112 may convey sound vibrations from the patient via the tubing, 104, the binaural apparatus 106, and the ear pieces 108. In one embodiment, the tubing 104 may convey the sounds picked up from the listening apparatus 102 to the binaural apparatus 106. In one embodiment, the tubing 104 may be a hollow tube with a Y-split at the end of the tubing connecting to the binaural apparatus 106. The tubing 104 may be a rubber material, a plastic material, a metal material, a polyurethane material, and so forth. In one example, the tubing 104 may be hollow with an air column within the hollow portion of the tubing 104.

The air column may conduct sound from the listening apparatus 102 to the binaural apparatus 106. The tubing 104 may be a flexible material such that the tubing 104 may flex or bend to allow the stethoscope to reach from the body of the patient to the ears of the medical professional. In another embodiment, the tubing 104 may include two sound paths inside an outer tubing to conduct sound to each ear of the medical professional to increase the fidelity and clarity of the sound. In another embodiment, the tubing 104 may include multiple tubes, such as two tubes, that separately connect to the junction piece 114. Each of the tubes may convey the sounds vibrations to different portions of the binaural apparatus 106 and ultimately to different earpieces 108. For example, a first tube may be connected to a first part of the binaural apparatus 106 and convey sound vibrations to a first one of the earpieces 108 and a second tube may be connected to a second part of the binaural apparatus 106 and convey sound vibrations to a second one of the earpieces 108.

The binaural apparatus 106 may be ear tubes that are positioned at an anatomically correct angle to properly fit into the ear canals of the medical professional. In one embodiment, the ear tubes may be hollow tubes (such as hollow metallic tubes) that are connected to the earpieces 108 on one end and the tubing 104 on the other end. The hollow tubes may include air columns that further convey sound from the tubing 104 to the earpieces 108.

In one example, the binaural apparatus 106 may include a first ear tube and a second ear tube. The first ear tube may connect to one of the Y-split ends of the tubing and the second ear tube may connect to the second of the Y-split ends. The binaural apparatus 106 may be a metal material, a plastic material, a rubber material, and so forth. In one example, the binaural apparatus 106 may be an aluminum alloy that is a strong and light weight material. In one embodiment, a tension of the ear tubes may be adjusted for individual fit and comfort by pulling the ear tubes apart to reduce the tension or squeezing them together (crossing them over) to increase the tension.

The earpieces 108 may be relatively small pieces of hollow material that are shaped to fit into the ear of a listener, such as a medical professional. In one embodiment, a first earpiece may be attached to the first ear tube and a second earpiece may be attached to the second ear tube. When the earpieces 108 are inserted into the ears of the listener, the earpieces 108 may convey the sound from the ear tubes to the ears of the listener. The earpieces 108 may be a silicone material, a plastic material, a polyurethane material, and so forth.

In one example, when the listening apparatus 102, the tubing 104, the binaural apparatus 106, and the earpieces 108 are connected together, as described above, the stethoscope 100 may use sound waves to transmit sound wave vibrations from a patient's body to the medical professional's ears. For example, as the body of the patient makes sounds, such as a heartbeat or valves opening and closing, the sounds produced or absence of sound may be low-frequency sounds or high-frequency sounds. The sounds may vibrate the bell 110 and/or the diaphragm 112 while the bell 110 and/or the diaphragm 112 are held against the body of the patient. From the listening apparatus 102, the sound wave vibrations may travel up to the tubes 104. The sound wave vibrations may then bounce off the sides of the tubes 104 and may be reflected up into the ear tubes of the binaural apparatus 106 and then to the earpieces 108.

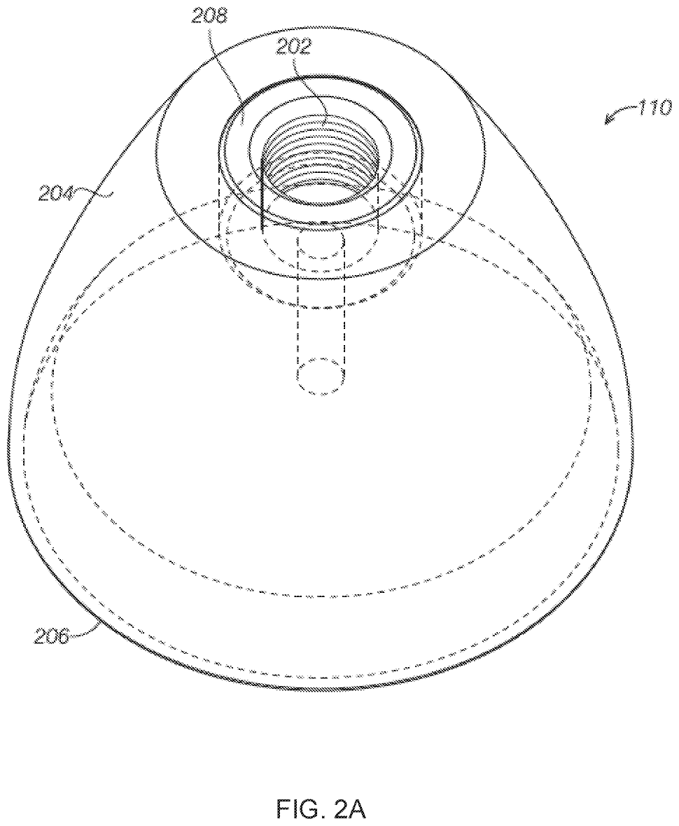

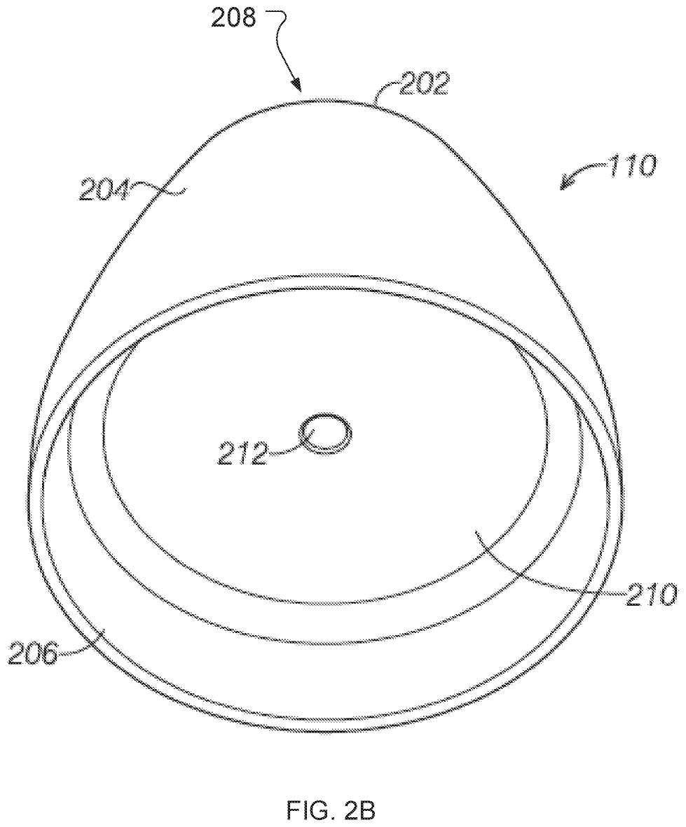

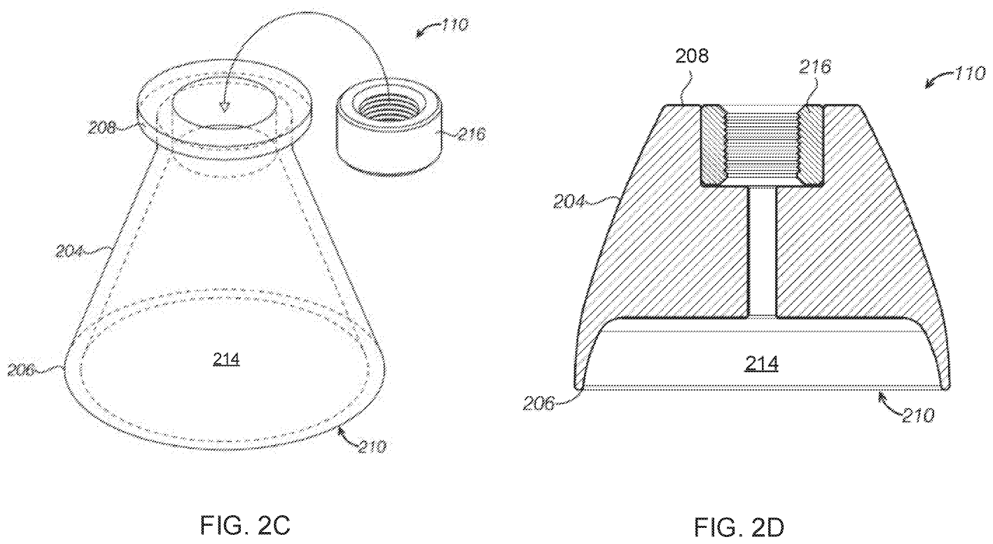

FIG. 2A illustrates a side perspective view of the bell 110 of the stethoscope 100 in FIG. 1, according to an embodiment. Some of the features in FIG. 2A are the same or similar to some of the features in FIG. 1 as noted by same reference numbers, unless expressly described otherwise. In one embodiment, the bell 110 may be a wood material. For example, the bell 110 may be a maple wood material, a spruce wood material, a cedar wood material, a mahogany wood material, a rosewood material, and so forth. The wood material of the bell 110 may have good acoustical properties to conduct the sound from the patient's appendage or organ. In one example, wood material may conduct sound in the longitudinal direction of the grain of the wood material. In another example, a dense wood material may reflect sound and can be configured to channel the sound reflections.

In another embodiment, the bell 110 may be a metal material (such as aluminum or copper), a plastic material, a glass material, or other sound conducting materials. In another embodiment, the bell 110 may include multiple materials.

In another embodiment, the bell may have an elongated tapering shape with a hollow interior. For example, the bell 110 may have a tapered conical shape. A base 206 of the bell 110 may have a larger diameter than a diameter of a top portion 208 of the bell 110. A tapered portion 204 of the bell 110 may taper upwardly and inwardly from the base 206 to the top portion 208 to form a cone-shaped bell. In one embodiment, the tapered portion 204 may taper at a angle between 60-75 degrees. In another embodiment, the bell 110 may include the wood material with the longitudinal direction of the grain extending from the base 206 to the top portion 208 to channel the sound from the patient's appendage or organ to the tubing 104 and ultimately the earpieces 108 of the stethoscope 100, as discussed above.

In one embodiment, the bell 110 may attach to the junction piece 114 of the stethoscope 100 in FIG. 1. In one example, the top portion 208 of the bell may include a connector 202. The connector 202 may be a hole with threads, ridges, and so forth that may connect with a corresponding connector of the junction piece 114. For example, when the connector 202 is integrated threads, the connector 202 may thread onto corresponding threads of the junction piece 114 to attach the bell 110 to the junction piece 114.

FIG. 2B illustrates a bottom perspective view of the bell 110, according to an embodiment. Some of the features in FIG. 2B are the same or similar to some of the features in FIGS. 1 and 2A as noted by same reference numbers, unless expressly described otherwise. As discussed above, the bell 110 may be configured to conduct the low-frequency sounds of an appendage or organ toward earpieces of the stethoscope 100.

In one embodiment, the bell 110 may include a partially hollow tapered cavity 210 where the partially hollow tapered cavity 210 extends inward from the base 206 of the bell 110. The partially hollow tapered cavity 210 may extend to the inner surface of the bell 110. In one example, the inner surface of the partially hollow tapered cavity 210 may be flat. In one embodiment, the partially hollow tapered cavity 210 may extend 0.5 inches to the inner surface plus or minus 0.1 inches. At a center of the inner surface, a channel or a conduit 212 may extend from the inner surface of the bell 110 to the top portion 208 or the connector 202 of the bell 110. In another embodiment, the channel or the conduit 212 may be 0.375 inches plus or minus 0.1 inches. In another embodiment, the channel or the conduit 212 may be 0.125 inches plus or minus 0.05 inches. The partially hollow tapered shape of the bell 110 may channel and direct the sound or vibrations from the sounds toward the top portion 208 of the bell 110 so that the sound may then be channeled by the stethoscope 100 from the bell 110 to the earpieces 108. In one example, the bell 110 may be shaped to pick up and conduct the low-frequency sounds of a patient's heart and/or lungs.

FIG. 2C illustrates a side perspective view of the bell 110, according to an embodiment. Some of the features in FIG. 2C are the same or similar to some of the features in FIGS. 1-2B as noted by same reference numbers, unless expressly described otherwise. As discussed above, the bell 110 may be configured to conduct the low-frequency sounds of an appendage or organ toward earpieces of the stethoscope 100.

In one embodiment, to conduct the sound, an interior of the bell 110 may be entirely hollow and tapered. The taper of the interior hollow portion of the bell 110 may correspond with an angle of the tapering of the exterior of the bell 110. The hollow tapered shape of the bell 110 may channel and direct the sound or vibrations from the sounds toward the top portion 208 of the bell 110 so that the sound or vibrations may then be channeled by the stethoscope 100 from the bell 110 to the earpieces 108. In one example, the bell 110 may be shaped to pick up and conduct the low-frequency sounds of a patient's heart and/or lungs.

In another embodiment, the top portion 208 of the bell 110 may include a threaded nut 216 that may be attached to the top portion 208 so that the bell 110 may be connected to the junction piece 114. In another example, the bell 110 may be a wood material and the threaded nut 216 may be a metal material. In another example, the bell 110 and the threaded nut 216 may be the same material. In another embodiment, an interior 214 of the bell 110 may be hollow and tapered.

FIG. 2D illustrates a cross-sectional view the bell 110 of the stethoscope 100 in FIG. 1, according to an embodiment. Some of the features in FIG. 2C are the same or similar to some of the features in FIGS. 1 and 2A-B as noted by same reference numbers, unless expressly described otherwise. In one embodiment, a diameter of the base 206 of the bell 110 may be 2 inches plus or minus 0.5 inches. In another embodiment, the height of the bell may be 2 inches plus or minus 0.5 inches. In another embodiment, the diameter of top portion 208 of the bell 110 may be 0.6 inches plus or minus 0.25 inches.

In another embodiment, the diameter of the base 206 of the bell 110 may be 4 inches plus or minus 1 inch. In another embodiment, the height of the base 206 of the bell 110 may be 4 inches plus or minus 1 inch. In another embodiment, the diameter of top portion 208 of the bell 110 may be 1.2 inches plus or minus 0.5 inches. The dimensions of the bell 110 are not intended to be limiting. In one example, the dimensions of the bell 110 may be proportional to each other where the overall proportions or size ratios remain the same but the overall size of the bell 110 may vary. In another embodiment, the diameters or height of the bell 110 may vary while the other dimensions remain the same.

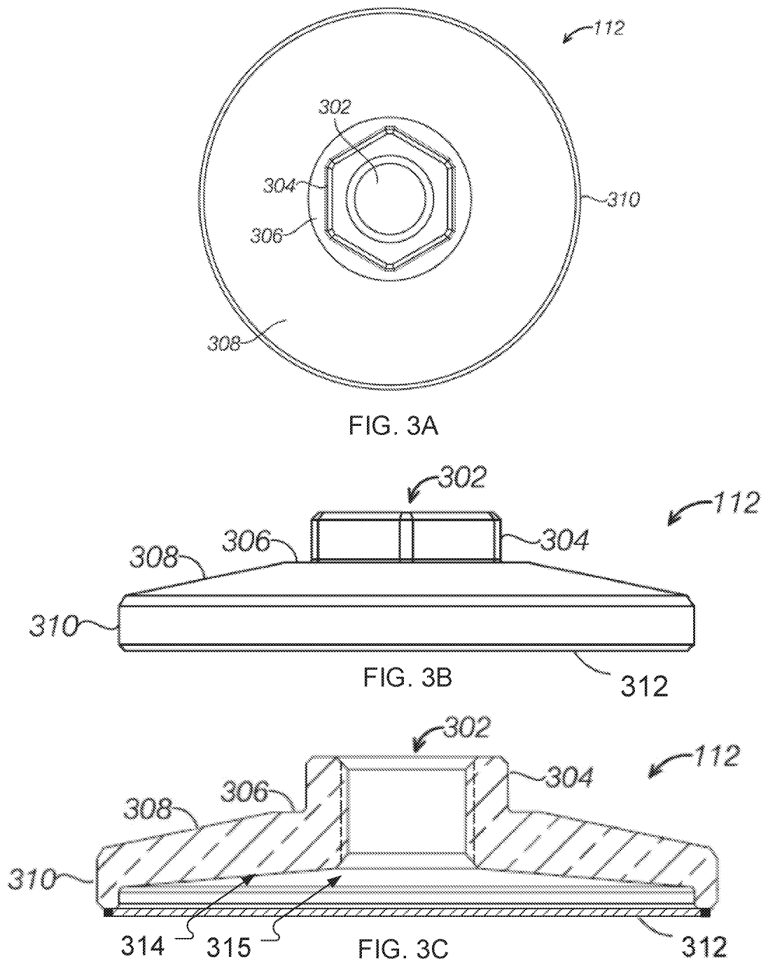

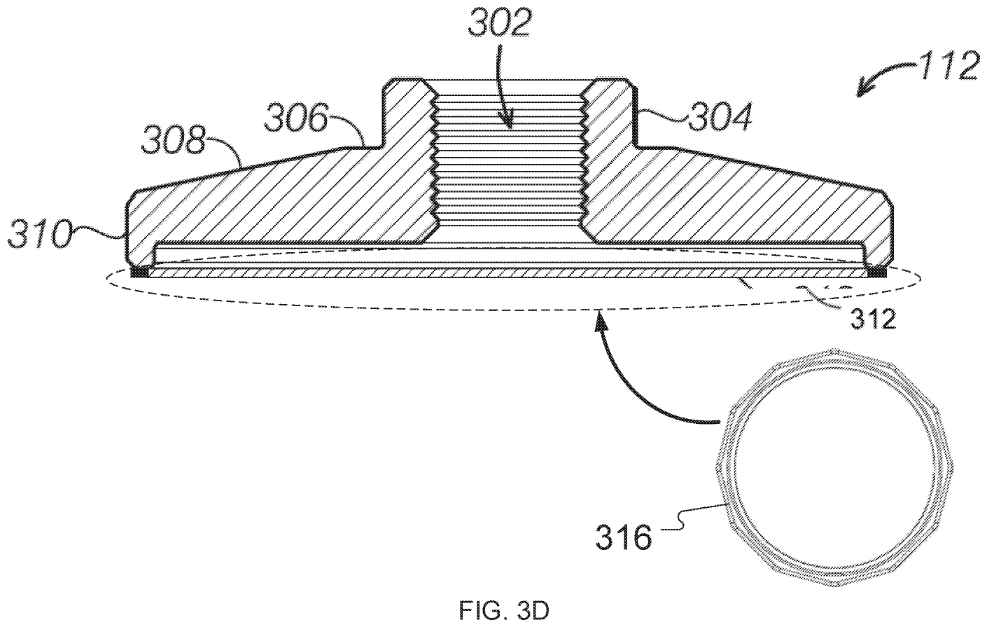

FIG. 3A illustrates a top view of the diaphragm 112 in FIG. 1, according to an embodiment. Some of the features in FIG. 3A are the same or similar to some of the features in FIG. 1 as noted by same reference numbers, unless expressly described otherwise. The diaphragm 112 may include a top portion 306, a tapered portion 308, and a base 310. The base 310 may be cylinder shaped with a defined height and uniform circular perimeter. The tapered portion 308 may extend upwardly at an angle and connect the base 310 to the top portion 306. The top portion 306 may be a flat surface of the diaphragm 112. The diaphragm 112 may include a connector 304 that may be configured to connect to the junction piece 114. In one embodiment, the connector 304 may include an opening to a cavity 302 of the diaphragm 112. The cavity 302 of the diaphragm 112 may extend from a center of the top side of the diaphragm 112 downward through a portion of a body of the diaphragm 112 to a disc 312 of the diaphragm 112 (as shown in FIG. 3C). In one embodiment, the disc 312 may be placed against a body of a patient to pick up sounds or vibrations from an appendage or organ of the patient. In one example, the disc 312 may be a metal material, a wood material, or other sound conducting material that may channel sound vibrations from the appendage or organ to the cavity 302. When the connector 304 is connected to the junction piece 114, the cavity may direct the sound vibrations to the junction piece 114, which may be conveyed to the earpieces 108, as discussed above.

FIG. 3B illustrates a side view of the diaphragm 112 with a disc 312, according to an embodiment. Some of the features in FIG. 3B are the same or similar to some of the features in FIGS. 1-3A as noted by same reference numbers, unless expressly described otherwise. In one embodiment, the diaphragm 112 may include a disc 312.

In one embodiment, the disc 312 may be attached to the bottom of the diaphragm 112 by a fastener, such as epoxy, hooks, loops, and so forth. In another embodiment, the disc 312 may be a metal material, a wood material, a plastic material, a glass material, and so forth. The disc 312 may be shaped to further channel the sounds from the appendage or organ by curving upward to a center point of the disc 312.

In one embodiment, the disc 312 may be attached to the bottom of the diaphragm 112 by a fastener, such as epoxy, hooks, loops, and so forth. In another embodiment, the disc 312 may be a metal material, a wood material, a plastic material, a glass material, and so forth. In one example, the disc 312 may be concavely curved so that the curved disc 312 curves towards the body of the diaphragm 112.

FIG. 3C illustrates a side view of the diaphragm 112 with a tapered cavity 314, according to an embodiment. Some of the features in FIG. 3C are the same or similar to some of the features in FIGS. 1-3B as noted by same reference numbers, unless expressly described otherwise.

In one embodiment, a bottom portion of the diaphragm 112 may include a tapered cavity 314 that tapers from the base 310 inwardly toward the cavity 302. In one example, the tapered cavity 314 may curve inward at approximately a 5 degree angle. In another embodiment, the tapered cavity 314 may curve inward at an angle between 1-25 degrees. When the disc 312 is a flat disc, the tapered cavity 314 may provide an air gap 315 between at least a portion of the disc 312 and a top of the tapered cavity 314. For example, except for where the disc 312 connects to the diaphragm 112 via a fastener, there may be the air gap 315 between the disc 312 and the top of the tapered cavity 315. In one example, the disc may be 0.005 inches thick and the air gap 315 between an inner surface of the disc 312 and the top of the tapered cavity 314 may be between 0.05 inches and 0.07 inches. In one example, the configuration of the diaphragm with the disc 312 and the air gap 315 may provide additional surface area for sound absorption to increase a clarify of the sound captured by the diaphragm 112.

FIG. 3D illustrates a side exposed view of the diaphragm 112 with a retaining ring 316, according to an embodiment. Some of the features in FIG. 3D are the same or similar to some of the features in FIGS. 1-3C as noted by same reference numbers, unless expressly described otherwise. In one embodiment, the connector 304 may be 0.5 inches in diameter plus or minus 0.2 inches. In another embodiment, the cavity 302 may be 0.375 inches in diameter plus or minus 0.1 inches. In another embodiment, an overall height of the diaphragm 112 may be 0.4 inches plus or minus 0.25 inches. In another embodiment, a thickness of the disc 312 may be 0.005 inches plus or minus 0.002 inches. In another embodiment, a diameter of the disc 312 may be 1.7 inches thick plus or minus 0.3 inches. The dimensions of the diaphragm 112 are not intended to be limiting. In one example, the dimensions of the diaphragm 112 may be proportional to each other where the overall proportions or size ratios remain the same but the overall size of the diaphragm 112 may vary.

In one embodiment, the diameters or height of the diaphragm 112 may vary while the other dimensions remain the same. In another embodiment, the disc 312 may be connected or fastened to the diaphragm 112 by a retaining ring 316. In one example, an interior surface of the disc 312 may abut or be approximate to an interior surface of the diaphragm 112 beneath the cavity 302. In another example, the disc 312 may sit or rest on a bottom surface of the diaphragm 112. The bottom surface of the diaphragm 112 may include a first set of threads that may mate with a second set of threads of the retaining ring 316 to hold the disc 312 against the diaphragm 112. In one example, the first set of threads may be female threads and the second set of threads may be male threads, or vice versa. In another example, the thread pitch of the first set of threads and/or the second set of threads may be 1.75″ OD×32 threads per inch. While, FIG. 3D illustrates the disc 312, the retaining ring 316 may similarly fasten the curved disc 312 in FIG. 3B to the diaphragm 112.

FIG. 4 illustrates the junction piece 114 connected to the tubing 104, the bell 110, and the diaphragm 112, according to an embodiment. Some of the features in FIG. 4 are the same or similar to some of the features in FIGS. 1-3C as noted by same reference numbers, unless expressly described otherwise. As discussed above, the stethoscope 100 in FIG. 1 may include the junction piece 114 that connects the tubing 104, the bell 110, and the diaphragm 112 together. For example, the junction piece 114 may include a first connector 402 extending from a first side of the junction piece 114, a second connector 404 extending from a second side of the junction piece 114, a third connector 406 extending from a bottom side of the junction piece 114, and a fourth connector 408 extending from the bottom side of the junction piece 114. The configuration of the connectors 402-408 of the junction piece 114 is not intended to be limiting. For example, the connectors 402-408 may extend from any of the sides or ends of the junction piece 114. The connectors 402-408 may be threads, ribs, clips, and so forth. The types of connectors 402-408 may correspond with connectors of the tubing 104, the bell 110, and the diaphragm 112, respectively.

In one embodiment, the first connector 402 may be a male thread that may be connected to a female thread of the connector 304 and partially extend into the cavity 302 of the diaphragm 112. In another embodiment, the second connector 404 may be a male thread that may be connected to a female thread of the connector 202 of the bell 110. In another embodiment, the third connector 406 may be a first barb that may be at least partially inserted into an interior of a first tube of the tubing 104. In another embodiment, the fourth connector 408 may be a second barb that may be at least partially inserted into an interior of a second tube of the tubing 104.

The connectors 402-408 of the junction piece 114 may enable the tubing 104, the bell 110, and/or the diaphragm 112 to be disconnected from the junction piece 114 to be replaced or switched with another part. For example, the bell 110 that has a first characteristic may be switched with another bell with a second characteristic. Diaphragms and/or tubing with different characteristics may also be interchanged. The characteristics may include sizes, shapes, dimensions, materials, and so forth.

In one embodiment, the first connector 402 may have an inner diameter of 0.25 inches and an outer diameter of 0.375 inches. In another embodiment, the length of the junction piece 114 between the first connector 402 and the second connector 404 may be 1.5 inches. In another embodiment, the distance between the third connector 406 and the fourth connector 408 may be 0.5 inches. In another embodiment, an inner diameter of the third connector 406 or the fourth connector 408 may be 0.17 inches. In another embodiment, the junction piece 114 may be a metal material, a plastic material, a wood material, and so forth. The dimensions, shape, size, or material of the junction piece 114 is not intended to be limiting. For example, as the dimensions, shape, size, or material of the bell 110, the diaphragm 112, and/or the tubes 104 change, the dimensions, shape, size, or material of the junction piece 114 may correspondingly change.

The disclosure above encompasses multiple distinct embodiments with independent utility. While these embodiments have been disclosed in a particular form, the specific embodiments disclosed and illustrated above are not to be considered in a limiting sense as numerous variations are possible. The subject matter of the embodiments includes the novel and non-obvious combinations and sub-combinations of the various elements, features, functions and/or properties disclosed above and inherent to those skilled in the art pertaining to such embodiments. Where the disclosure or subsequently filed claims recite “a” element, “a first” element, or any such equivalent term, the disclosure or claims is to be understood to incorporate one or more such elements, neither requiring nor excluding two or more such elements.

Applicant(s) reserves the right to submit claims directed to combinations and sub-combinations of the disclosed embodiments that are believed to be novel and non-obvious. Embodiments embodied in other combinations and sub-combinations of features, functions, elements and/or properties may be claimed through amendment of those claims or presentation of new claims in the present application or in a related application. Such amended or new claims, whether they are directed to the same embodiment or a different embodiment and whether they are different, broader, narrower or equal in scope to the original claims, are to be considered within the subject matter of the embodiments described herein.

1. An apparatus, comprising:
a wood bell configured to capture a low-frequency vibration from a surface of a body, wherein the wood bell comprises:
a first base having a first diameter;
a first tapered portion extending upwardly from the first base and tapering inwardly, wherein at least a portion of the first tapering portion is at least partially hollow;
a first top portion having a second diameter and connected to a top of the first tapered portion, wherein the first base, the first tapered portion, and the first top portion form an elongated cone shape;
a first channel extending from a bottom the base to the at least partially hollow portion of the first tapered portion;
a first connector connected to the first top portion;
a diaphragm configured to capture a high-frequency vibration from the surface of the body, wherein the diaphragm comprises:
a second base having a third diameter, wherein at least a portion of the second base comprising a disc along a bottom surface of the second base;
a second tapered portion extending upwardly from the second base and tapering inwardly;
a second top portion having a fourth diameter and connected to a top of the second tapered portion;
a second channel extending from the second top portion to the disc;
a second connector connected to the first top portion;
a junction piece comprising:
a third connector connected to the first connector;
a fourth connector connected to the second connector;
a fifth connector connected to a first end of tubing;
the tubing comprising a column of air configured to convey at least one of the low-frequency vibration or the high-frequency vibration to a second end of the tubing;
a binaural apparatus having a first end connected to the second end of the tubing, wherein the binaural apparatus is configured to convey at least one of the low-frequency vibration or the high-frequency vibration to a second end of the binaural apparatus; and
a first earpiece connected to the second end of the binaural apparatus, wherein the first earpiece is configured to convey at least one of the low-frequency vibration or the high-frequency vibration to a first ear of a listener.

2. The apparatus of claim 1, wherein:
the tubing comprises a first tube and a second tube; and
the junction piece comprises a sixth connector, wherein the first tube is configured to connect to the fifth connector and the second tube is configured to connect to the sixth connector.

3. The apparatus of claim 1, further comprising a second earpiece connected to the second end of the binaural apparatus, wherein the second earpiece is configured to convey at least one of the low-frequency vibration or the high-frequency vibration to a second ear of the listener.

4. The apparatus of claim 1, wherein the first connector integrated into the first top portion.

5. The apparatus of claim 1, wherein the at least partially hollow portion of the first tapered portion is entirely hollow.

6. The apparatus of claim 1, wherein the at least partially hollow portion of the first tapered portion extends from the first base of the wood bell inward to a defined distance below that is below the first top portion of the wood bell.

7. The apparatus of claim 6, wherein the at least partially hollow portion of the first tapered portion include a conduit that extends from the defined distance below the first top portion to the top of the first top portion.

8. The apparatus of claim 1, wherein the wood bell is a maple wood material, a spruce wood material, a cedar wood material, a mahogany wood material, or a rosewood material.

9. The apparatus of claim 1, wherein:
a diameter of the first base is between 1.5 inches and 2.5 inches;
a height of the wood bell is between 1.5 inches and 2.5 inches; and
a diameter of the first top portion is between 0.35 inches and 0.85 inches.

10. The apparatus of claim 1, wherein:
a diameter of the first base is 2 inches;
a height of the wood bell is 2 inches; and
a diameter of the first top portion is 0.6 inches.

11. The apparatus of claim 1, wherein:
a diameter of the second connector is between 0.3 inches and 0.7 inches;
a diameter of the second channel is between 0.275 inches and 0.475 inches;
a height of the diaphragm is between 0.15 inches and 0.65 inches; and
a thickness of the disc is between 0.04 inches and 0.08 inches.

12. The apparatus of claim 1, wherein:
a diameter of the second connector is 0.5 inches;
a diameter of the second channel is 0.375 inches;
a height of the diaphragm is 0.4 inches; and
a thickness of the disc is 0.06 inches.

13. An apparatus, comprising:
a bell configured to capture a low-frequency vibration from a surface of a body, wherein the bell comprises:
a first base having a first diameter;
a first tapered portion extending upwardly from the first base and tapering inwardly, wherein at least a portion of the first tapering portion is hollow;
a first top portion having a second diameter and connected to a top of the first tapered portion, wherein the first base, the first tapered portion, and the first top portion form an elongated cone shape;
a first channel extending from a bottom of the first base to the hollow portion of the first tapered portion; and
a first connector connected to the first top portion; and
a diaphragm configured to capture a high-frequency vibration from the surface of the body, wherein the diaphragm comprises:
a second base having a third diameter, wherein at least a portion of the second base comprising a disc along a bottom surface of the second base;
a second tapered portion extending upwardly from the second base and tapering inwardly;
a second top portion having a fourth diameter and connected to a top of the second tapered portion;
a second channel extending from the second top portion to the disc; and
a second connector connected to the first top portion.

14. The apparatus of claim 13, further comprising a junction piece comprising:
a third connector connected to the first connector;
a fourth connector connected to the second connector; and
a fifth connector connected to tubing.

15. The apparatus of claim 13, wherein the first tapered portion is entirely hollow.

16. The apparatus of claim 13, wherein the wood bell is a maple wood material, a spruce wood material, a cedar wood material, a mahogany wood material, or a rosewood material.

17. The apparatus of claim 13, wherein the disc is curved convexly.

18. An apparatus, comprising:
a bell configured to capture a low-frequency vibration from a surface of a body, wherein the bell comprises:
a base having a first diameter;
a tapered portion extending upwardly from the base and tapering inwardly;
a top portion having a second diameter and connected to a top of the first tapered portion, wherein the base, the tapered portion, and the top portion form an elongated cone shape; and
a channel extending from a bottom of the base to the tapered portion.

19. The apparatus of claim 18, wherein the bell is a wood material with a grain of the wood material extending longitudinally from the base to the top portion.

20. The apparatus of claim 18, wherein at least a portion of the tapering portion is hollow.
